(19) United States
(12) Patent Application Publication
(10) Pub. No.: US 2024/0382437 A1
(43) Pub. Date: Nov. 21, 2024

(54) TREATMENT OF ACUTE TISSUE INJURY

(71) Applicant: OBVIA PHARMACEUTICALS LTD., Madison, WI (US)

(72) Inventors: William FAHL, Madison, WI (US); Torsten GOESCH, Singapore (SG); Hannah Rebecca GOESCH, Gruenwald (DE); Sarah Ricarda GOESCH, Gruenwald (DE)

(21) Appl. No.: 18/578,618

(22) PCT Filed: Jul. 14, 2022
(86) PCT No.: PCT/EP2022/069810

§ 371 Date: Jan. 11, 2024

Related U.S. Application Data
(60) Provisional application No. 63/222,663, filed on Jul. 16, 2021.

(51) Int. Cl. A61K 31/145; A61P 29/00

(52) U.S. Cl. CPC A61K 31/145; A61P 29/00

(57) ABSTRACT

The present invention is directed to the use of the compound Prc-210. or a pharmaceutically acceptable acid addition salt thereof. for use in the treatment of an inflammation in a tissue of a subject, which inflammation is caused by an acute tissue injury.

### FIELD OF THE INVENTION

The present invention is directed to the use of the compound Prc-210 or a pharmaceutically acceptable acid addition salt thereof, for use in the treatment of an inflammation in a tissue of a subject, which inflammation is caused by an acute tissue injury.

### BACKGROUND OF THE INVENTION

End-stage renal failure causes greater than 1.2 million deaths annually in the world. Kidney transplantation is the preferred treatment for patients with end-stage renal disease. Over 90,000 kidney transplants are performed each year in the world.

The transplant process, itself, induces significant cellular and organ injury to the kidney, which reduces short and long-term survival of the organ. The four primary insults to a kidney during an allograft transplant are i) reactive oxygen and nitrogen species (ROS and RNS)-induced damage from systemic shock and ischemia before and during removal of the organ from the donor, ii) ROS and RNS induced damage during cold storage (‘cold-ischemia’), iii) ROS-induced damage upon implant (‘re-perfusion injury’) trigger post-allograft-transplant inflammation, which triggers the innate immune response and antibody-mediated rejection (ABMR), and iv) immune response damage to the transplanted organ in the short-and long-term (Sellares et al, Am J Transplant 2012; 12:388-399).

Neutrophils and macrophages migrate into the allograft transplant within 6 hr of reperfusion and stimulate chemokine synthesis in resident dendritic cells that then activate T lymphocytes and recruit adaptive immune cells. Once these immune cells infiltrate the proximal tubule epithelial cells, they produce myeloperoxidase in neutrophils and nicotinamide adenine dinucleotide phosphate (NADPH) oxidase in macrophages, which contribute to local free radical production. These inflammatory processes lead to an activation of the complement pathways and further cell remodeling and lysis in the kidney allograft.

ABMR can occur as a result of either, or both, preformed alloantibody against the graft or through the de novo development of donor-specific antibody (dnDSA) (Hidalgo et al, Am J Transplant 2009;9:2532-41).

The acute (min/days), transitioning to chronic (days/weeks), inflammatory response within the allograft kidney, with continuous production of free-radicals (ROS and RNS) and inflammatory cytokines and chemokines, can establish a severe, self-perpetuating response that causes long-term kidney organ failure (Loupy et al; Nature Rev Nephrology; 2012;8:348-57).

To better understand cellular, molecular and immunological pathways involved in the pathogenesis of kidney allograft inflammation and rejection, we developed and characterized a rat model that replicates most of the clinical criteria of the acute immune response, ABMR and kidney organ loss

(Huang et al; Am J Transplant. 2014;14:1061-1072). This model has been used to evaluate a number of novel post-allograft transplant strategies.

The two currently acknowledged approaches for reducing the severity of the acute and long-term immune response against the kidney allograft are: i) to increase the chance of finding a cross-matched donor, and ii) to remove preexisting antibodies against the kidney allograft using desensitization protocols (Stegall et al; Am J Transplant. 2006; 6:346-351).

### SUMMARY OF THE INVENTION

The present invention is based on the appreciation that a reduction in the severity of the immune response, e.g., reducing the cell damage in kidney allografts can be achieved by using the compound PrC-210 (3-(methylamino)-2-(methylaminomethyl) propane-1-thiol), a free radical-scavenger (Peebles et al; Radiat Res. 2012; 178:57-68), to directly and chronically scavenge and inactivate inflammation-generating, and inflammation-generated, free radicals e.g. within the newly transplanted allograft kidney. This new, inventive, approach would significantly enhance the two existing strategies, and would in itself provide a new pathway to reduce acute, post-transplant, kidney cell damage.

PrC-210 is the prototype of a new family of direct-acting, small molecule aminothiol free radical-scavengers; it has no measurable nausea/emesis nor hypotension side effects (Soref et al; Int J Rad Onc Biol Phys. 2012; 82: e701-707).

In two previous rodent kidney transplant studies, PrC-210 was shown to suppress free-radical-induced kidney damage induced during i) 30 hr cold storage (Verhoven et al Transplantation Direct. 2020;6: e578) and ii) reperfusion injury upon implant (Bath et al; Transplantation Direct. 2019;5: e549-555) to background levels, thus removing two substantial sources of injury to the transplanted kidneys during the transplantation process. The PrC-210 molecule has also been shown to suppress free radical-induced injury in several other organ settings. Thus, it has now been realized that PrC-210 can protect an allograft against oxidative stress that is generated by both i) cellular-and ii) antibody-mediated rejection processes that produce free radicals as a byproduct.

Accordingly, in an aspect of the present invention, there is the compound Prc-210 of the formula:

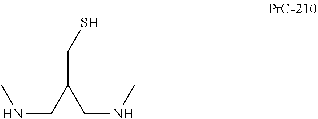

or a pharmaceutically acceptable salt thereof, for use in the prevention or treatment of an inflammation in a tissue of a subject, which inflammation is caused by an acute injury.

In an aspect of the present invention, there is the use of the compound Prc-210 of the formula:

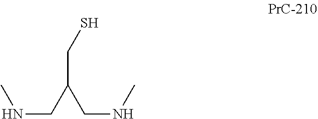

or a pharmaceutically acceptable salt thereof, for the manufacture of a medicament for the prevention or treatment of an inflammation in a tissue of a subject, which inflammation is caused by an acute injury.

In an aspect of the present invention, there is a method for the prevention or treatment of an inflammation in a tissue exposed to an acute injury, said method comprising administering to a subject the compound PrC-210

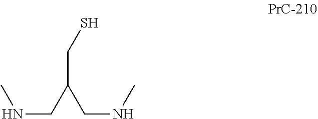

or a pharmaceutically acceptable acid addition salt thereof.

In an aspect of the present invention, there is the compound PrC-210

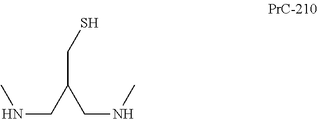

or a pharmaceutically acceptable acid addition salt thereof, for use in the prevention of an organ transplant rejection.

In an aspect of the present invention, there is the compound PrC-210 for use in the treatment of Acute Radiation Syndrome (ARS).

In an aspect of the present invention, there is a method for the treatment of an inflammation in a tissue of a subject, which inflammation is caused by an acute tissue injury, whereby the compound PrC-210

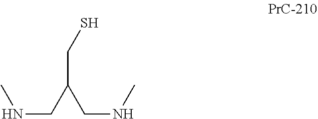

or a pharmaceutically acceptable acid addition salt thereof, is administered to a subject in need of such treatment.

### DESCRIPTION OF THE FIGURES

FIG. 1 shows an experimental schematic of the kidney surgery and PrC-210 administration times for the BN rat kidney transplants into LEW rats. LT (left), RT (right), MTD (Maximum Tolerated Dose), IP (intraperitoneal).

FIG. 2 shows the histology of (A) BN kidney upon removal from BN rat. (B,C,D) BN kidneys 20 hr following transplant into LEW rats. “20 hr” control kidneys (B,C) were flushed with room temperature UW Solution prior to transplant, and +PrC-210 kidneys (D) were flushed with room temperature UW Solution containing 30 mM PrC-210 prior to transplant, and recipient LEW rat also received three intraperitoneal systemic PrC-210 doses in the eight hours following transplant. Panel B red arrows highlight tubulitis pathology, and Panel C yellow arrows highlight peritubular capillaritis pathology. Panel E shows summed tubulitis and peritubular capillaritis severity scores for three kidneys in each of the indicated groups.

FIG. 3 shows (A,B,C) kidney histology 20 hr following transplant. Operator randomly collected sample images of renal tubules from each of the indicated kidney groups in panels A,B,C. Then, using an ImageJ macro, H/E images (e.g., FIG. 3 panels A-C) were analyzed, and above-threshold pink or blue pixels were selected and enumerated, and their ratio was then calculated and plotted to provide an estimate of kidney white blood cell infiltration.

FIG. 4 shows the effects of PrC-210 administration upon kidney function 20 hr after transplant of the BN kidney into a LEW rat. Rats either received no PrC-210 (“20 Hr”) or three IP PrC-210 injections in the 20 hr period following transplant (“20 Hr +PrC-210”), before recipient rat was euthanized and serum was collected. P values are indicated.

FIG. 5 shows the caspase activity in post-transplant kidneys. Kidney supernatant activated caspase 3,7 activity was measured enzymatically in a 60 min reaction.

FIG. 6 shows the effect of PrC-210 administration upon serum and kidney homogenate cytokine levels 20 hr after transplant of the BN kidney into a LEW rat. TIMP-1, TNF-alpha and MIP-3A/CCL20 levels were determined by ELISA analysis. P values are indicated.

FIG. 7 shows a schematic summarizing PrC-210 efficacy in suppressing each of the three organ insults that occur during kidney transplantation.

FIG. 8 shows a schematic summarizing PrC-210 efficacy in suppressing Caspase 1 in plasma of ICR mice when PrC-210 is given before or after lethal radiation with 8.68 Gy.

FIG. 9 shows a schematic summarizing PrC-210 efficacy in suppressing Caspase 8 in brains of ICR mice when PrC-210 is given before lethal radiation with 8.68 Gy.

### DETAILED DESCRIPTION OF THE INVENTION

In one aspect of the invention, the compound Prc-210 is for use as herein described, before or after the exposure to an acute injury.

In another aspect, there is the compound Prc-210 for use in the prevention of the development of an inflammation in a tissue exposed to the acute injury.

In another aspect of the invention, a tissue exposed to an acute injury is selected from kidney tissue, liver tissue, heart tissue, brain, spine or neuronal tissue, vascularized extremities, bone marrow tissue, intestine tissue and lung tissue.

In another aspect of the invention, a tissue inflammation as herein described is acute tissue inflammation.

In another aspect of the invention, an acute tissue inflammation as herein described may be induced by an acute traumatic event.

In another aspect of the invention, an acute tissue injury as herein described is a secondary tissue injury.

In another aspect of the invention, an acute tissue injury as herein described is fibrosis. Hence, an aspect of the invention is the compound PrC-210 for use in the prevention or treatment of fibrosis caused by an acute tissue injury.

In another aspect of the invention, an acute tissue injury as herein described is present in a transplanted tissue received by a subject.

In another aspect of the invention, an acute tissue injury as herein described is induced during a transplantation procedure.

In another aspect of the invention, an acute tissue injury as herein described is due to surgery.

In another aspect of the invention, an acute tissue injury as herein described is due to a trauma.

In another aspect of the invention, an acute tissue injury as herein described is due to a burn or major wound.

In another aspect of the invention, an acute tissue injury as herein described is caused by a microbial or virus infection.

In another aspect of the invention, an acute tissue injury as herein described is caused by stroke, myocardial infarction or pulmonary embolism.

In another aspect of the invention, an acute tissue injury as herein described is caused by traumatic brain injury, spinal cord injury or traumatic injuries of extremities or central organs.

In another aspect of the invention, an acute tissue injury as herein described is caused by radiation, for example, wherein the acute tissue injury is caused by Acute Radiation Syndrome (ARS).

In another aspect of the invention, an acute tissue injury as herein described is caused by ischemia or ischemic reperfusion injury.

In another aspect of the invention, the compound Prc-210 is administered systemically before, during or after a tissue injury as herein described has occurred.

In another aspect of the invention, the compound Prc-210 is for prophylactic use before or after an acute injury in the prevention of secondary damage caused by an acute injury.

In another aspect of the invention, the compound Prc-210 is for use in fibrosis such as lung fibrosis, liver fibrosis, cardiac fibrosis or kidney fibrosis.

In another aspect of the invention, the compound Prc-210 is for use as herein described, in the prevention of an organ transplant rejection in a donor who provides a transplant organ.

In another aspect of the invention, the compound Prc-210 is for use as herein described, in a preservation solution for the transport of a donor organ.

In another aspect of the invention, the compound Prc-210 is for use in a subject who has received a transplant organ from a transplant donor.

In another aspect of the invention, the compound Prc-210 is for use in the suppression of an inflammation-associated organ damage post-transplant.

According to the invention, upon treatment as described above, the histological score of tissue damage in the exposed tissue is reduced. The exposed tissue may be selected from kidney tissue, bone marrow tissue, brain and spinal tissue, heart tissue, muscle tissue, nerve tissue, jejunum tissue and lung tissue.

Further according to the invention, upon treatment, the level of an inflammation marker is reduced. The marker may be selected from the group of serum TNF alpha; NOS2; serum albumin alpha; infiltration of mononuclear cells into the damaged tissue; and chemokine synthesis in resident dendritic cells.

The invention also involves, upon treatment, the increased activated caspase levels such as Caspase 1, 3/7 and 8 to reduced or normalized physiological levels.

The invention also involves that, upon treatment, MIP-3a/CCL20 levels are upregulated and/or regulatory T-cells, in particular TREG cells, are activated and/or that the level of tissue inhibitor of metalloproteinase-1 (TIMP-1) is reduced.

According to the invention, acute inflammation is reduced.

The invention also leads to normalization of inflammation-associated cell death.

The invention also reduces secondary tissue damage, and also fibrosis.

According to the invention, the tissue to be treated is transplanted tissue received by the subject and the injury is associated with the transplantation; the injury is caused by exposure to an acute radiation or nuclear event; the subject is undergoing a surgery causing the tissue injury; the subject suffered from a trauma causing the tissue injury; the subject suffered from burns or major wounds causing the tissue injury; the tissue injury is caused by a microbial or virus infection; the tissue injury is caused by stroke, myocardial infarction or pulmonary embolism; the tissue injury is caused by traumatic brain injury, spinal cord injury or traumatic injuries of extremities or central organs; or the tissue injury is caused by ischemia or ischemic reperfusion injury.

In one aspect of the invention, the compound PrC-210 herein disclosed is used for the treatment of Acute Radiation Syndrome (ARS). The compound PrC-210 herein disclosed may also be used for the prevention of Acute Radiation Syndrome (ARS). For example, the compound PrC-210herein disclosed may be administered to a subject intended to undergo radiation therapy. Accordingly, the compound PrC-210 may be administered to a subject prior to the subject being exposed to radiation therapy.

The invention may involve systemic administration of an effective amount of the compound at an effective time before, during or after the damaging event causing the tissue injury. In one aspect, the tissue is protected by prophylactic administration of the compound.

The wording “inflammation in a tissue” as used herein means inflammation caused by local immune, vascular and inflammatory cell responses to an injury or infection of a tissue.

The wording “tissue” as used herein means a structure with similar types of specialized cells that perform unique functions in the body of multicellular organisms, such as connective tissue, epithelial tissue, muscle tissue, and nerve tissue. Epithelial tissues line the surfaces and cavities of the body, protecting the body from dehydration and mechanical damage. Connective tissue connects and binds separate tissues and organs together. The connective tissue is composed of few cells, embedded in a matrix, which is secreted by the cells in the connective tissue. Muscular tissue is involved in controlling internal body functions such as digestion, respiration, urination as well as external body functions such as movement of body parts. Nerve tissue coordinate both internal and external functions of the body with the different stimuli.

The wording “inflammation caused by an acute injury” as used herein means inflammation caused by a sudden body damage. Such inflammation may occur in any organ of the human body which has been subjected by a trauma or a physical damage of an organ.

The wording “organ” as used herein means any organ of the human body such as kidney, bone marrow, jejunum (small intestine), colon, lung, liver, and brain.

The wording “organ tissue” as used herein means any organ tissue of the human body such as kidney tissue, bone marrow tissue, jejunum (small intestine) tissue, colon tissue, lung tissue, liver tissue, and brain tissue.

The wording “secondary tissue injury” as used herein means destructive and self-propagating biological changes in cells and tissues that lead to their dysfunction or death over hours to weeks after the initial insult (the “primary injury”). In most contexts, the initial injury is usually mechanical.

The wording “fibrosis” as used herein means the development of fibrous connective tissue as a reparative response to injury or damage. Fibrosis may refer to the connective tissue deposition that occurs as part of normal healing or to the excess tissue deposition that occurs as a pathological process. When fibrosis occurs in response to injury, the term “scarring” is used. Examples of fibrosis in the human body is lung fibrosis (pulmonary fibrosis), liver fibrosis (liver cirrhosis), cardiac (heart) fibrosis, mediastinal fibrosis, retroperitoneal cavity fibrosis, bone marrow fibrosis, skin fibrosis, and scleroderma or systemic sclerosis.

Pulmonary fibrosis may occur as a result of long standing infections such as tuberculosis or pneumonia. The condition can also be caused by exposure to occupational hazards such as coal dust or the genetic condition cystic fibrosis.

Liver fibrosis or Cirrhosis refers to the scar tissue and nodules that replace liver tissue and disrupt liver function. The condition is usually caused by alcoholism, fatty liver disease, hepatitis B or hepatitis C.

Cardiac fibrosis or heart fibrosis are areas of the heart that have become damaged due to myocardial infarction may undergo fibrosis.

Mediastinal fibrosis is fibrosis characterized by calcified fibrosis of the lymph nodes, which can block respiratory channels and blood vessels.

Retroperitoneal cavity fibrosis refers to fibrosis of the soft tissue in the retroperitoneum, which contains the aorta, kidneys and numerous other structures.

Bone marrow fibrosis or myelofibrosis is scarring in the bone marrow that prevents the normal production of blood cells in the bone marrow.

Skin fibrosis is scar tissue that forms on the skin in response to injury is referred to as a keloid.

Scleroderma or systemic sclerosis is an autoimmune disease of the connective tissue that primarily affects the skin but can also involve other organs such as the kidneys, heart and lungs.

The wording Acute radiation syndrome (ARS), also known as radiation sickness or radiation poisoning, is a collection of health effects that are caused by being exposed to high amounts of ionizing radiation in a short period of time. Examples of such radiation poisoning is radiation caused by a nuclear event.

The wording “transplanted tissue” or “transplanted organ” as used herein means a tissue or an organ which has been taken from a subject (the donor) and been transplanted into a patient in need of a tissue or an organ.

The wording “allograft” as used herein means a surgical transplant of tissue (or organs) between genetically different individuals of the same species;

The wording “prophylactic therapy” as used herein means that the compound Prc-210 or a pharmaceutically acceptable acid addition salt thereof, is used for the prevention inflammation in a tissue as described herein. This wording also encompasses the prevention of fibrosis as herein described, and the prevention of rejection of a transplanted organ in a subject who has received a transplant organ from a transplant donor.

The wording “suppression of an inflammation-associated organ damage post-transplant” as used herein means that the compound Prc-210 is used to inhibit or reduce inflammation of an organ in a patient who has received an organ from a donor of said organ.

The wording “the compound PrC-210” as used herein means the compound:

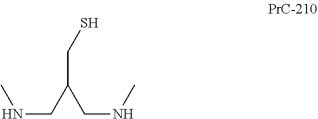

or a pharmaceutically acceptable acid addition salt thereof. This compound and the manufacture thereof is disclosed in i.a. U.S. Pat. No. 7,314,959 B2.

### EXAMPLES

### Example 1

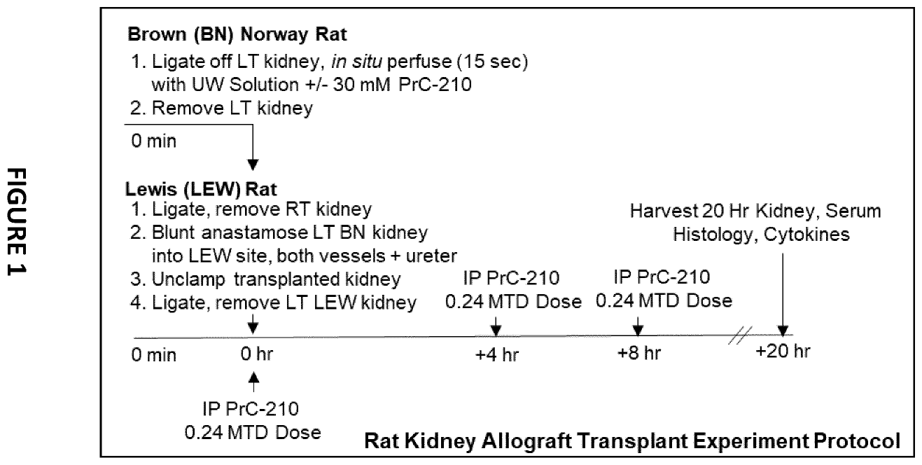

To determine PrC-210 efficacy in suppressing inflammation-generated damage to a transplanted, allograft organ, we used an allograft rat kidney transplant model that largely eliminated the induction of the ischemia and reperfusion insults in transplant, and created only the third inflammation insult by allograft transplanting a kidney from a Brown strain (BN) rat into a recipient Lewis strain (LEW) rat (FIG. 1). PrC-210 was administered both pre-and post-implantation to determine its protective effect in suppressing any inflammation-associated damage to the transplanted BN kidney in the LEW rat. Brown Rat kidneys were flushed with UW solution containing PrC-210 and immediately transplanted into syngeneic Lewis Rat recipients. Ischemic time was virtually eliminated. Immediately following implant, and for 8 hr following kidney implant, recipient rats received systemic PrC-210 injections at doses that would enable continuous free radical-scavenging within the transplanted kidney. Transplanted kidneys and blood plasma were then harvested 20 hr following transplant to enable measurement of both PrC-210-conferred i) suppression of inflammatory by-products and ii) kidney protection.

### Example 2

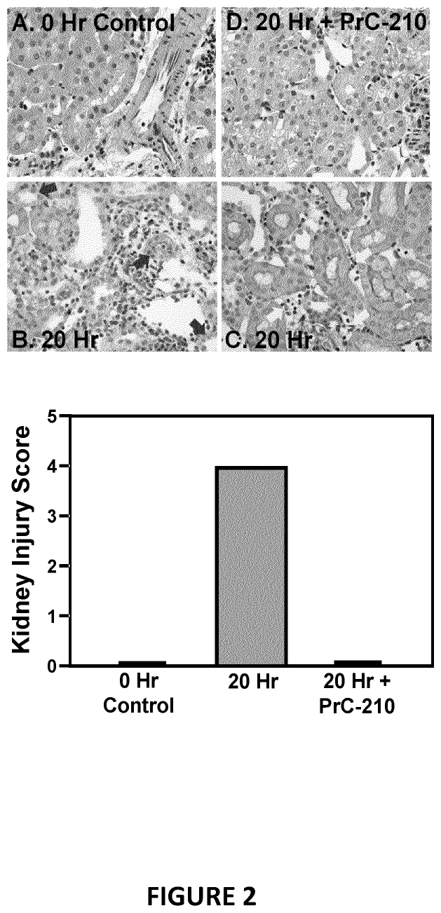

At 20 hr post-transplant, histology of the transplanted BN kidneys (FIG. 2, panels A-D) treated with UW Solution alone clearly showed increased Banff scores for tubulitis and peritubular capillaritis (red and yellow arrows); the summed pathology scores are shown in panel E. BN kidneys perfused with PrC-210-containing UW Solution and receiving post-implant intraperitoneal systemic doses of PrC-210 (FIG. 2, D) clearly showed suppressed inflammatory pathology to the kidney and suppressed Banff scores for tubulitis and peritubular capillaritis equal to Banff scores of the “0 Hr” control BN group (FIG. 2, E).

### Example 3

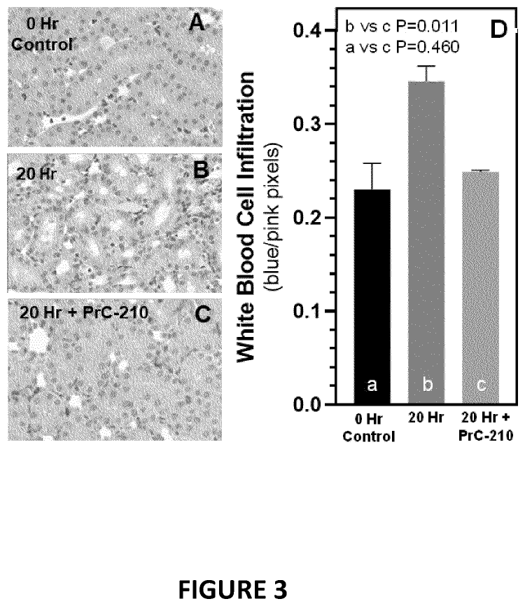

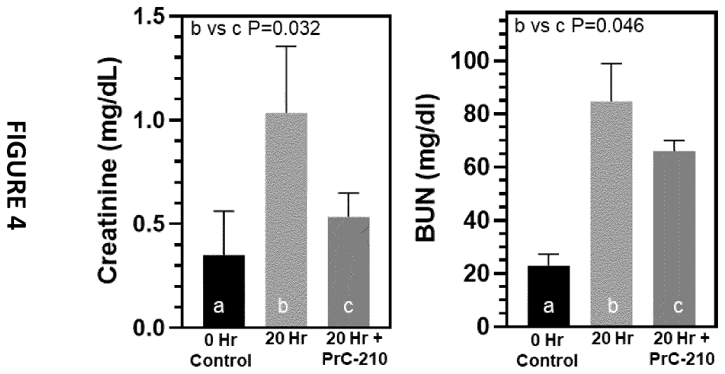

“20 hr” histology of the BN kidneys flushed with UW Solution only showed a significantly reduced thickness of the renal tubule brush border epithelium (FIG. 3, B) in comparison to the “0 hr” control BN kidneys just removed from a BN rat (FIG. 3, A). The histology of BN kidneys receiving PrC-210via both the perfusing UW Solution and intraperitoneal injections clearly showed a preserved integrity of the renal tubular brush border (FIG. 3 C).

The same blinded histology sections were examined for mononuclear white cell infiltration. BN kidneys at 20 hr flushed with UW Solution only showed a significant increase in the number of blue nuclei (scored as blue pixels), which reflects the infiltration of mononuclear white blood cells. In contrast, mononuclear infiltration in the BN kidneys flushed and treated with PrC-210 was significantly suppressed versus the untreated BN kidneys (P=0.011) and the Inflammatory Infiltration Score was statistically the same as the 0 hr control group (FIG. 3, D).

### Example 4

Serum creatinine and serum BUN were also measured to assess function in the BN allograft kidneys 20 hr after transplant (FIG. 4). Administration of PrC-210 conferred significant reductions in both the creatine (P=0.032) and BUN (P=0.046) kidney damage markers.

### Example 5

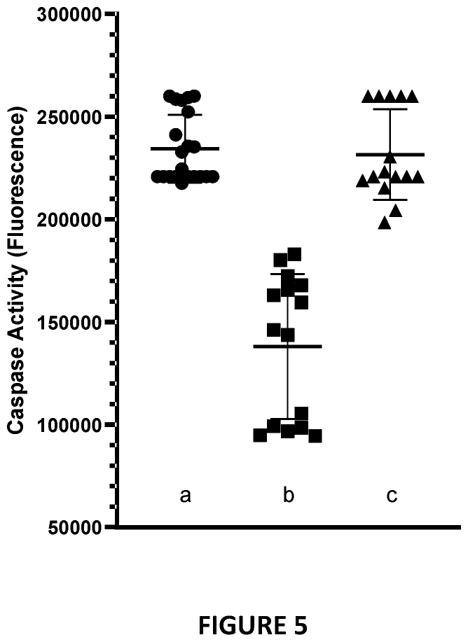

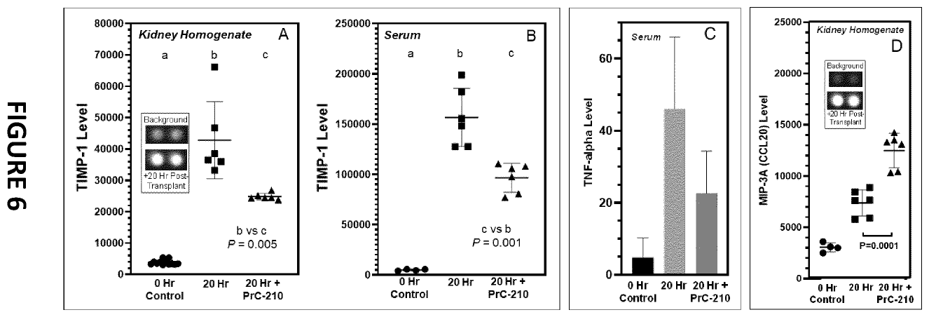

Levels of activated caspase in BN kidney homogenates were significantly reduced in BN allograft kidneys that were not exposed to PrC-210 treatment during the 20 hr following transplant (FIG. 5). Perfusion of BN kidneys with PrC-210-containing UW Solution and transplant into Lewis rats that received systemic PrC-210 doses resulted in the same activated caspase activity to that seen in “O hr” control kidneys, which is indicative of less kidney damage after non-ischemic kidney transplantation.

### Example 6

In screening experiments, kidney homogenate supernates from 0 hr controls and 20 hr no treatment kidneys were screened with the Proteome Profiler 29 cytokine array to detect altered, inflammation-associated, cytokine and chemokine expression levels 20 hr post-transplant. As shown in the two FIG. 6 microarray insets, changes were seen in TIMP-1, TNF-alpha and MIP-3a/CCL20. Individual ELISA plates were then used to quantify these changes in kidney homogenates and sera, now including rats treated with PrC-210 as well. Both TIMP-1 and TNF-alpha levels were increased 20 hr post-transplant, and in both cases, their levels were decreased in the presence of PrC-210 (FIG. 6, A-C). MIP-3a/CCL20 levels were increased at 20 hr, but they increased significantly higher in PrC-210-treated rats (FIG. 6D).

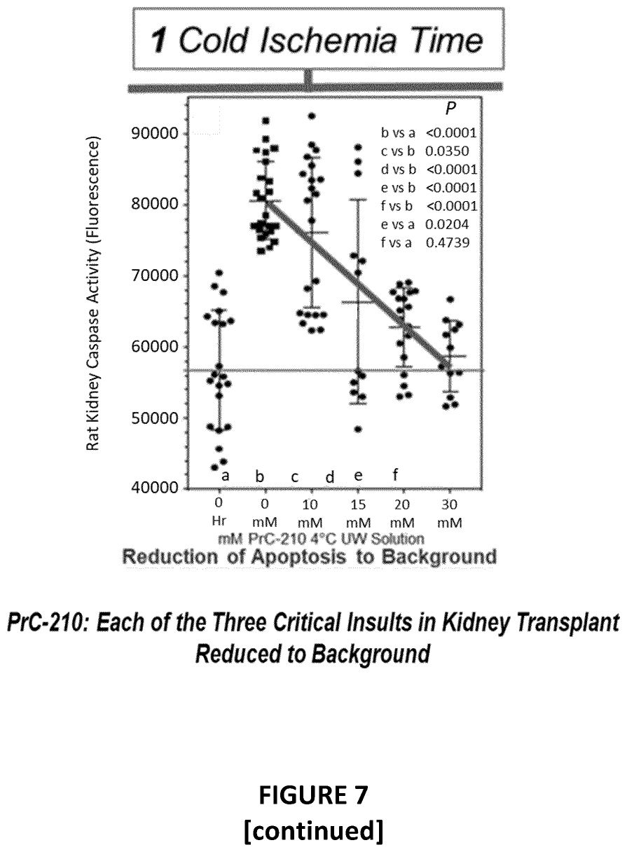

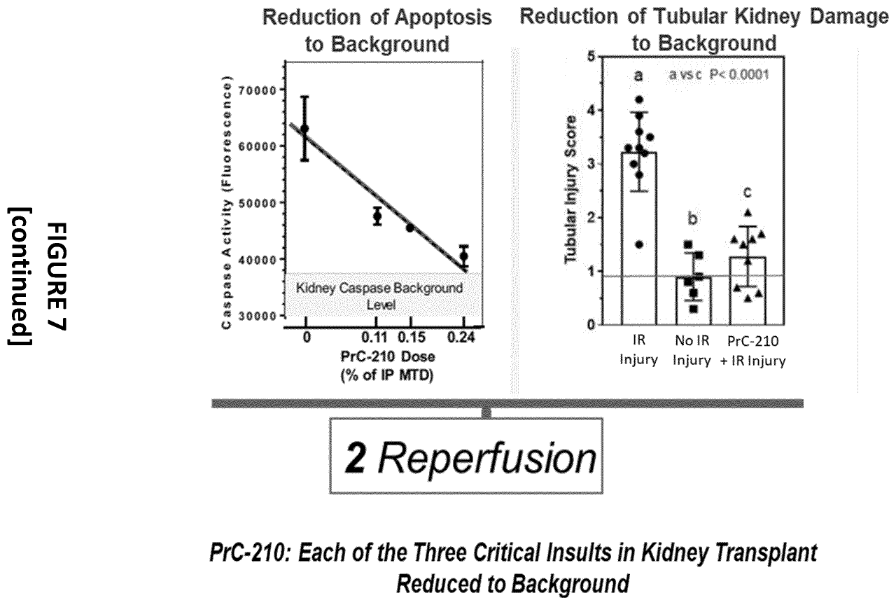

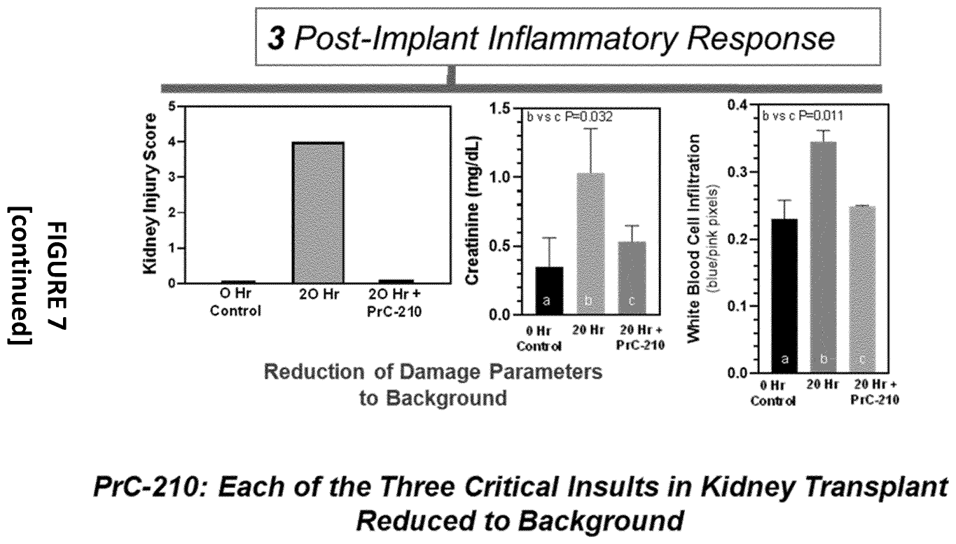

FIG. 7 summarizes the PrC-210 efficacy in suppressing each of the three organ insults that occur during kidney transplantation.

### Example 7

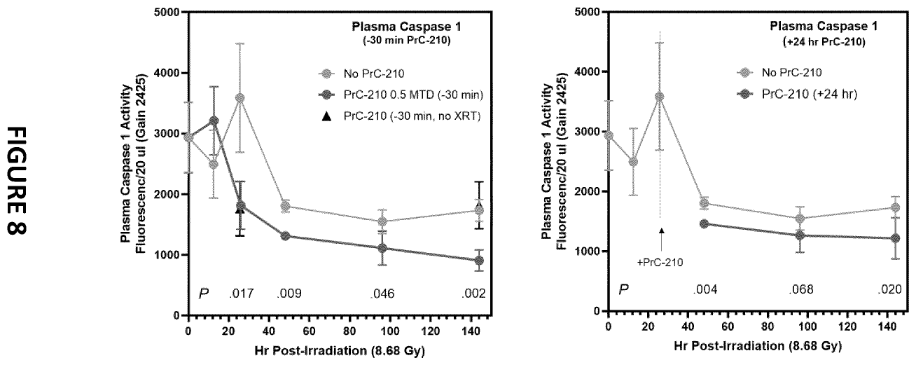

FIG. 8 shows a 6-day suppression by PrC-210 treatment of Caspase 1 in plasma after lethal radiation, which is support for a long-term inhibition of the inflammasome. Caspase 1 is a central activator of acute inflammation in neutrophilic cells, which recruits both T-cells and monocytes and activates apoptosis.

### Example 8

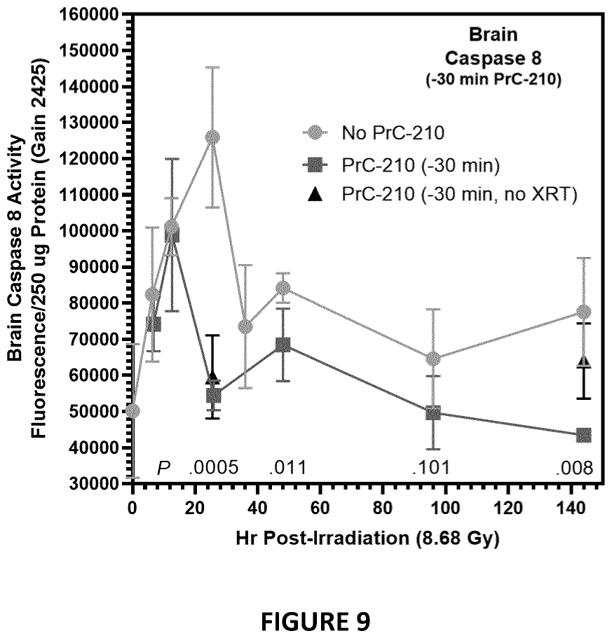

Caspase 8 inhibition (FIG. 9) in the brain after acute injury, such as radiation, is support for less TNF-α activation, and TRL—and FAS receptor upregulation in the brain. All three biomarkers get upregulated after acute injury, such as radiation or traumatic brain injury, and lead to an upregulation of Caspase 8. This supports that inflammation, such as activation of microglia is normalized after PrC-210. The effect on Caspase 8 lasts for at least six days, which is support for a long-term effect of PrC-210 in inhibition of inflammation.

1. A method for the of treatment of an inflammation in a tissue of a subject, wherein the inflammation is caused by an acute tissue injury, comprising administering compound PrC-210 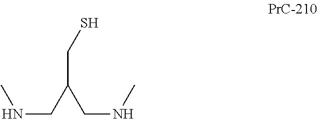
or a pharmaceutically acceptable acid addition salt thereof, to a subject in need of such treatment.

2. The method according to claim 1, wherein the inflammation is present after acute injury in tissue selected from kidney tissue, liver tissue, heart tissue, brain tissue, spine tissue, neuronal tissue, tissue of the vascular extremities, bone marrow tissue, intestine tissue (jejunum tissue) and lung tissue.

3. (canceled)

4. The method according to claim 1, wherein the treatment is preventive therapy.

5. The method according to claim 1, wherein the inflammation caused by an acute tissue injury is a secondary tissue injury.

6. The method according to claim 1, wherein the acute tissue injury leads to fibrosis.

7. The method according to claim 1, wherein the acute tissue injury is in a transplanted tissue received by a subject.

8. The method according to claim 1, wherein the acute tissue injury is induced during a transplantation procedure.

9. The method according to claim 1, wherein the acute tissue injury is due to surgery.

10. The method according to claim 1, wherein the acute tissue injury is due to a trauma.

11. The method according to claim 1, wherein the acute tissue injury is due to a burn or major wound.

12. The method according to any one of claims claim 1, wherein the acute tissue injury is caused by a microbial or virus infection.

13. The method according to any one of claims claim 1, wherein the acute tissue injury is caused by stroke, myocardial infarction or pulmonary embolism.

14. The method according to any one of claim 1, wherein the acute tissue injury is caused by traumatic brain injury, spinal cord injury or traumatic injuries of extremities or central organs.

15. The method according to any one of claims claim 1, wherein the acute tissue injury is caused by ischemia or ischemic reperfusion injury.

16. The method according to any one of claims claim 1, wherein the compound Prc-210 is administered systemically before, during or after the tissue injury.

17. The method according to any one of claim 1, wherein said treatment is prophylactic therapy in prevention of secondary damage from an acute injury.

18. The method according to claim 6, wherein the fibrosis is lung fibrosis, liver fibrosis, cardiac fibrosis, kidney fibrosis, mediastinal fibrosis, retroperitoneal cavity fibrosis, bone marrow fibrosis, skin fibrosis, scleroderma or systemic sclerosis.

19-22. (canceled)

23. A method of treatment of Acute Radiation Syndrome (ARS), comprising administration of compound PrC-210 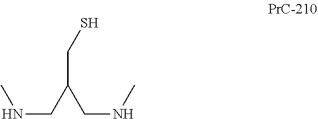
or a pharmaceutically acceptable acid addition salt thereof, to a subject in need of such treatment.

24. The method according to claim 23, wherein the treatment is preventive treatment.

25. A method of supressing an inflammation-associated organ damage post-transplant, comprising administration of compound PrC-210 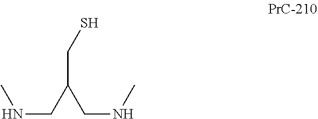
or a pharmaceutically acceptable acid addition salt thereof, to a subject in need of such treatment.

26. (canceled)
